(19) United States
(12) Patent Application Publication
(10) Pub. No.: US 2005/0107801 A1
(43) Pub. Date: May 19, 2005

(54) Apparatus for surgical instrument location

(76) Inventors: Brian Davies, London (GB); Justin Cobb, London (GB)

Correspondence Address: WALLENSTEIN WAGNER & ROCKEY, LTD, 311 SOUTH WACKER DRIVE, 53RD FLOOR, CHICAGO, IL, 60606, US

(21) Appl. No.: 10/495,998

(22) PCT Filed: Nov. 1, 2002
(86) PCT No.: PCT/GB02/04925

(30) Foreign Application Priority Data
Nov. 19, 2001 (GB) 0127658.3

(51) Int. Cl. A61B 17/58

(52) U.S. Cl. 606/96

(57) ABSTRACT

An apparatus for surgical instrument location comprises a generally cylindrical tube (10,12,14) one end of which preferably includes teeth (20) for securing the tube to an area of bone to be prepared for an implant. An insert (60) fits within the tube and has a longitudinal aperture (64) the lateral edges of which define an operational area for an instrument inserted through the aperture. Means are provided for selectively positioning the aperture at a desired longitudinal and rotational position within the tube. An outer end (66) of the aperture is shaped, and forms a camming surface which, in co-operation with a cam follower on the surgical instrument, varies the longitudinal position of the instrument within the tube as the instrument is moved laterally across the operational area. Different sizes and shapes of tube may be provided, along with different inserts for each tube, allowing the surgeon to select an appropriate combination. The apparatus provides an inexpensive passive guidance system for use in minimal access surgery. The apparatus further protects surrounding tissues from debris.

The present invention relates to apparatus for enabling a surgeon to maintain the position of instruments within a precise, known, area during a surgical procedure. The invention is particularly although not exclusively for use in surgical procedures requiring minimal access.

Joint replacement (arthroplasty) and other orthopaedic operations normally require extensive incision of the skin along with a substantial amount of soft tissue dissection. This is normally necessary to allow the area of bone or joint to be visualised, to allow the skeleton to be orientated, and to allow the soft tissues to be retracted in order to protect them from the cutting tools used in the preparation of the bone surface.

Having performed the initial incisions, the surgeon typically inserts retractors to pull back the soft tissue and then applies a series of procedures, often with aligning jigs to maintain the orientation of the instruments used. Once the bone surfaces have been prepared, the implant is inserted, and the soft tissues restored.

The conventional need for extensive soft tissue dissection has a number of serious drawbacks:
It is time consuming.
It is risky, as it allows the tissues to be exposed to the air, and body fluids and debris to contaminate the operating room.
It causes the patient pain, and extends the length of hospital stay as the patient recovers from the pain, swelling and stiffness of the incision through the skin fascia and muscle.
Alignment of the instruments needed for preparing the bone for the implant is difficult. Normally a sequence of jigs is used, with an error in the positioning of one jig compounding subsequent errors in the alignment of the implant relative to the prepared bone surface. Also, the anatomy is often distorted by the bone exposure process and the subsequent reaction of soft tissue; this makes alignment even more difficult.
Because of these alignment and other errors, the quality of the interface between the prepared bone surface and the implant is frequently less than perfect.

In recent years, a number of minimal access approaches have been developed which use manual or automated navigational aids, or robotic assistance, to improve the accuracy of the procedure. There still, however, remains a need for an easy to use and relatively inexpensive method for the surgeon accurately to locate the bone-preparation instruments. The protection of the patient's soft tissues, and the operating room generally, from the debris inevitably produced by the bone preparation procedure also remains a limiting factor.

It is an object of the present invention at least to alleviate the difficulties of the prior art.

It is a further object of the invention to provide an easy to use and relatively inexpensive apparatus allowing surgical instruments to be kept within a precisely-defined operational area, as required by the surgeon.

It is a further object of the invention to protect the patient's soft tissues, the surgeon, and the operating room itself from the debris created by the bone preparation procedure.

According to a first aspect of the invention there is provided an apparatus for surgical instrument location comprising an elongate open-ended tube, and an insert for insertion into the tube, the insert having a longitudinal aperture formed therein the lateral edges of which define an operational area for an instrument inserted through the aperture.

According to a further aspect of the invention there is provided a surgical instrument location system comprising:
(a) a plurality of open-ended tubes of differing sizes or cross sections;
(b) a corresponding plurality of inserts for insertion into the tubes, each insert having a longitudinal aperture formed therein the lateral edges of which define an operational area for an instrument inserted through the aperture.

The invention in its various forms provides an easy to use and inexpensive passive guidance system, and tissue protection system, which is particularly useful for minimal access surgery, for example for endoluminal arthoplasty. Use of the system allows a reduction in the size of the incision needed for joint replacement, provides protection for the surrounding area during the operation, and improves the accuracy of the procedure.

The apparatus of the present invention (preferably used with standard intraluminal jigs) allows technical procedures such as bone preparation to be performed by drills, saws or milling tools. The tool orientation is defined by the orientation of the tube, with the instrument being aligned, and the area of operation defined, by apertures or grooves within the insert.

Although the apparatus of the invention, in its preferred embodiment, is designed for use in bone-preparation procedures, the invention in its broadest form is not limited to such use. In other embodiments, the apparatus may be used for procedures on ligaments or even on soft tissue. In such uses, the tube would normally be held stationary by an external jig.

The tubes are of predefined shape, and may accordingly be tracked (that is, located in three-dimensions) by any standard surgical navigation system.

The invention will be carried into practice in a number of ways, and several specific embodiments will now be described, by way of example, with reference to the accompanying drawings, in which:

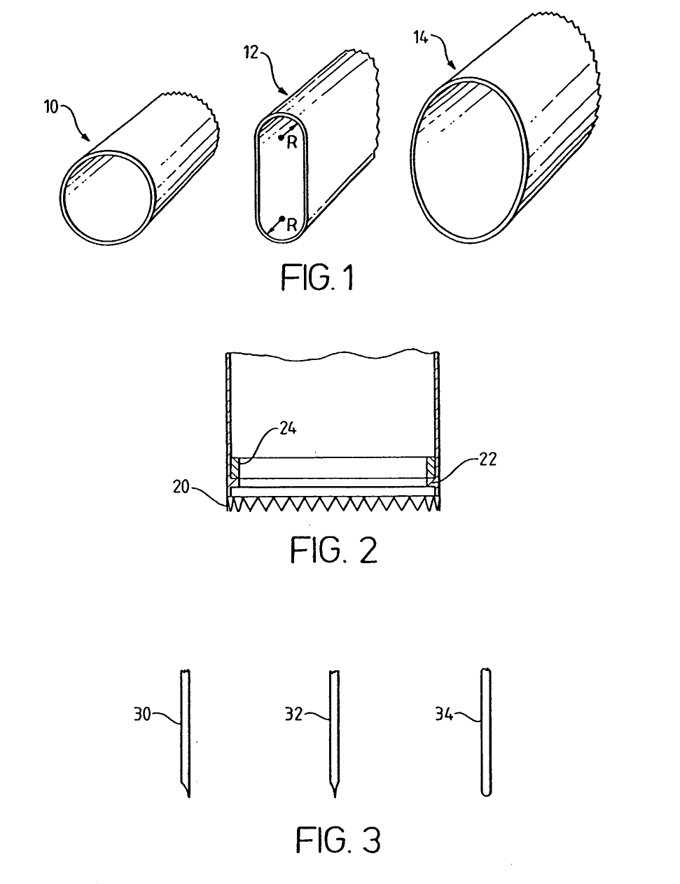

FIG. 1 shows the general shape of three embodiments of the apparatus of the present invention;

FIG. 2 shows the teeth at the end of the tube;

FIG. 3 shows possible tube wall cross-sections;

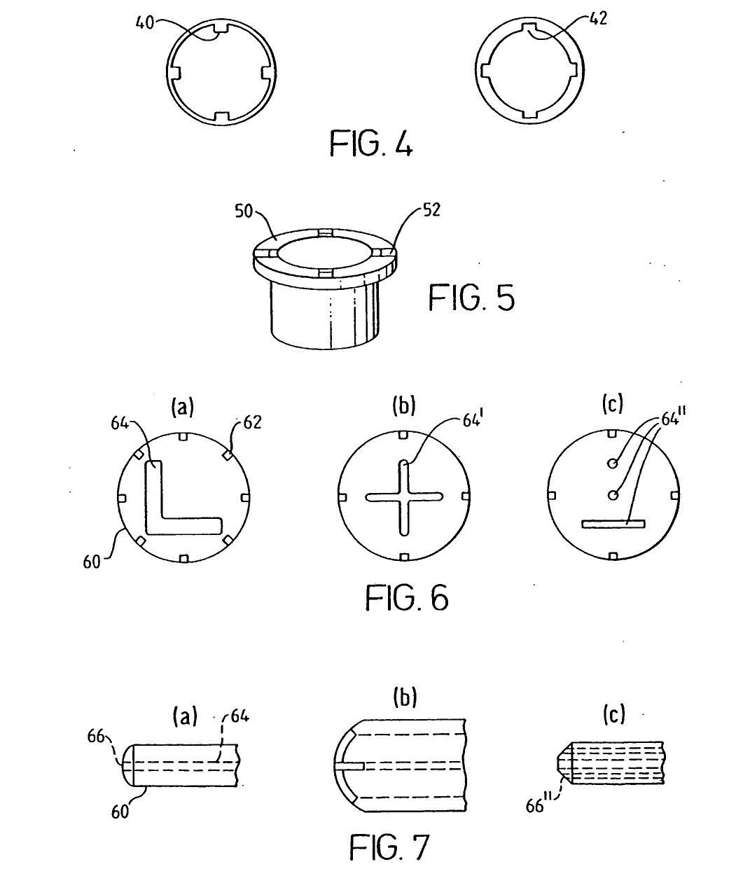

FIG. 4 shows two possible internal axial constraining features;

FIG. 5 shows a possible rim fitting;

FIGS. 6a to 6c show sections through various possible fixtures, for use within the tube;

FIG. 7 shows partial side views of the fixtures of FIGS. 6a to 6c;

FIG. 8 shows the external features for securing, in use, a joint capsule;

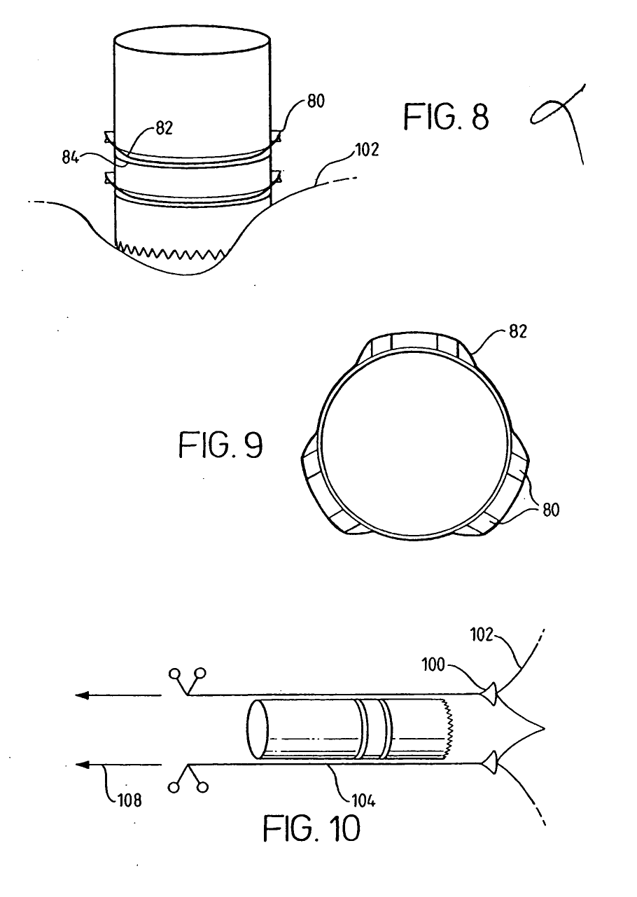

FIG. 9 is a cross-section through the tube, in use, showing the capsule secured by a gaiter;

FIG. 10 shows the way in which the capsule is pulled up over the tube;

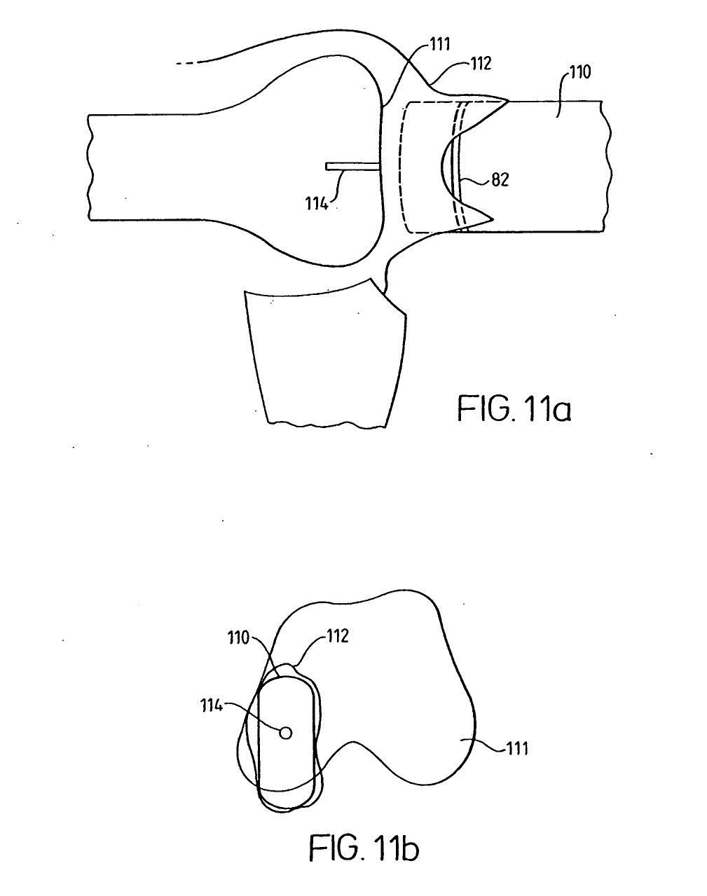

FIGS. 11a and 11b are, respectively, longitudinal and cross-sections through a knee joint, showing the apparatus in use;

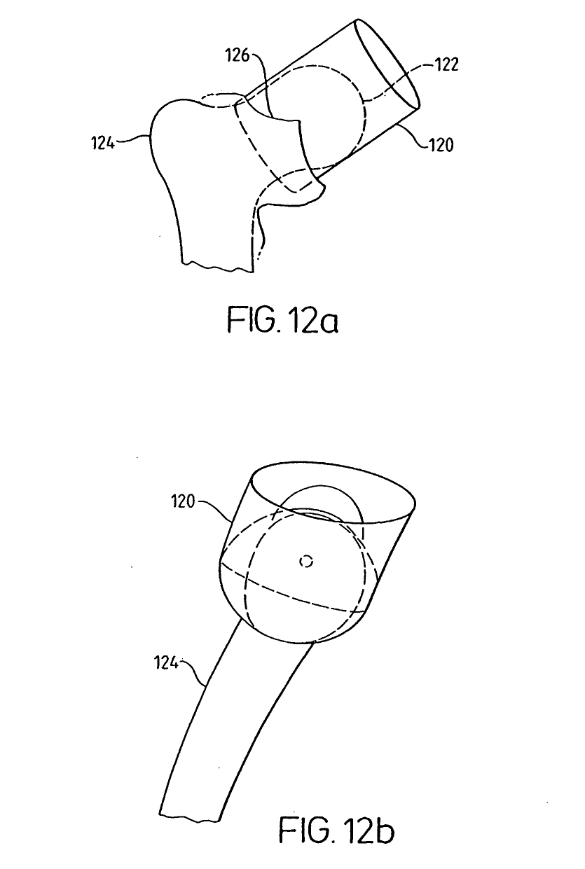

FIGS. 12a and 12b are, respectively, front and side views of a hip joint, showing the apparatus in use;

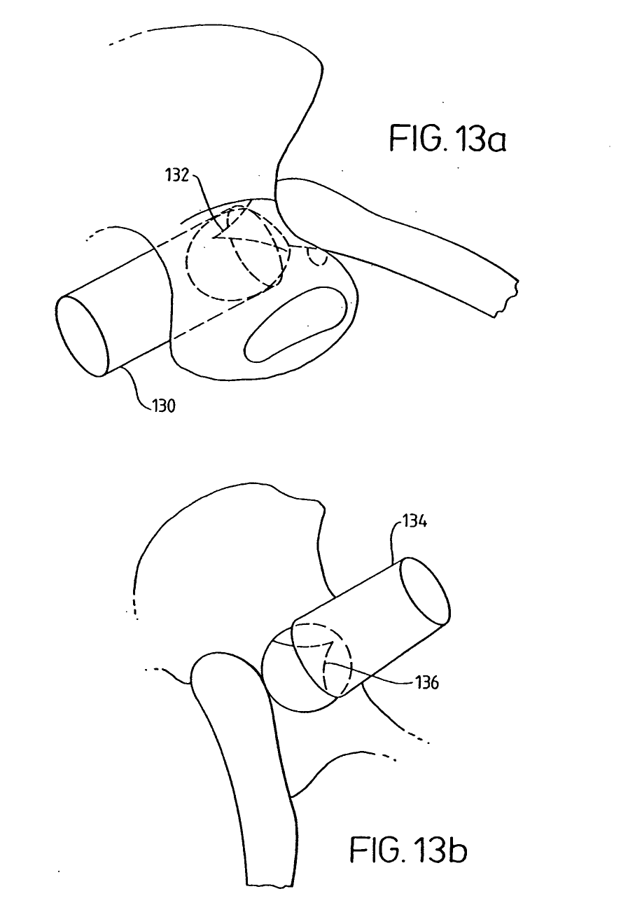

FIGS. 13a and 13b show the apparatus in use on a hip joint; and

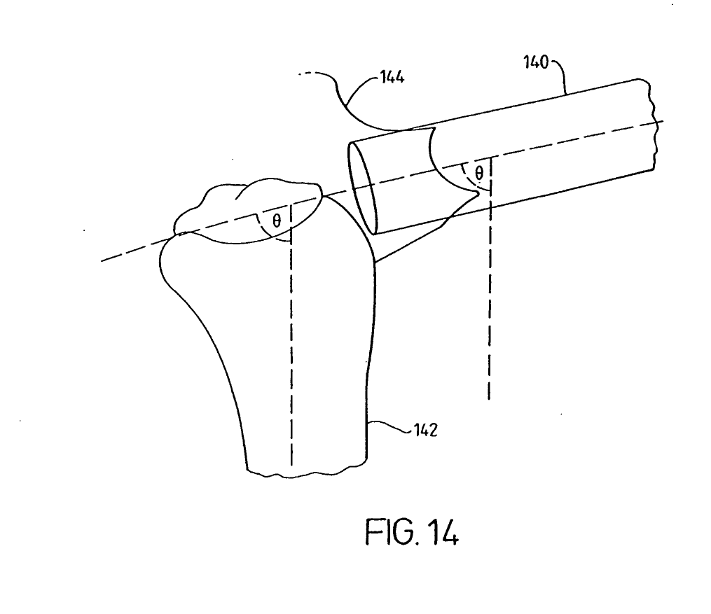

FIG. 14 shows the apparatus in use for a knee and tibial preparation.

The apparatus of the present invention includes a tube (various embodiments of which are illustrated in FIGS. 1 to 5) and an insert that fits within the tube (various embodiments of which are shown in FIGS. 6 and 7). Typically, both the tube and the inserts are manufactured of an inert plastics material, but it would also be possible for both or either to be metal.

FIG. 1 shows three possible tube shapes, namely a right circular thin-walled cylinder 10, a cylinder 12 having parallel sides that are joined by radiused sections, and an oval cylinder 14. A variety of different tubes are preferably provided, of differing shapes and cross-sections, allowing the surgeon simply to select the tube that is most appropriate at the time, given the procedure to be undertaken and the size of the patient.

One of the tube ends is preferably formed with a series of fixed or sprung teeth 20, allowing that end of the tube to be hammered into or otherwise secured on an exposed bone surface. Alternatively, as shown in FIG. 3, the teeth may be omitted and the end of the wall may be sharpened, either on one face as shown by reference numeral 30, or on both faces as shown by reference numeral 32. Alternatively, as shown at 34, if the wall is sufficiently thin the end may not need any special preparation, as it may naturally be sharp enough to hold the tube steady against the bone.

As shown in FIG. 4, the inner tube surface may be formed with a longitudinally-extending series of lugs 40, or slots 42, providing engagement for an appropriately-shaped insert to be slid down inside the tube. There may be more or fewer lugs or slots than is shown in FIG. 4. Alternatively, as shown in FIG. 5, the tube may have an outwardly-directed rim 50 at one end, with slots or location points 52 being provided on that rim. In such a case, the corresponding insert (not shown) will have an enlarged head portion, typically with positioning lugs which fit into the slots 52, thereby holding the insert secure against rotation.

Various types of insert suitable for use with the left hand embodiment of FIG. 4 will now be described, with reference to FIGS. 6 and 7. In each case, it will be understood that the locating slots in the inserts could be replaced with axial lugs, allowing the insert to be used with the right-hand embodiment of FIG. 4. Alternatively, a suitably-sized head (not shown) could be provided for use with the embodiment of FIG. 5.

FIGS. 6a and 7a show one type of insert that can be used with the tube of FIG. 4. The insert is an elongate solid plastics member 60, of a suitable shape to be received closely within the tube, and having longitudinal slots 62 which are arranged to be received over the lugs 40 of the tube. The number of slots 62 may be the same as the number of lugs 40, or alternatively more slots 62 may be provided: where the tube cross-section has some rotational symmetry, this allows the insert to be positioned in a number of different rotational positions with respect to the tube.

Extending the entire length of the insert is a custom-shaped slot 64, which may be of any appropriate size and shape depending upon the cuts that need to be made into the bone to prepare the bone surface. The outer end 66 of the insert (that is, the end that faces away from the bone in use) is domed, or is formed with some other custom shape. In use, a cutting instrument (not shown) is passed down through the slot 64, and is located longitudinally by means of a stop on the instrument which abuts the outer end 66. As the cutting proceeds, the stop moves across the surface of the outer end; the surface therefore effectively acts as a cam, forcing the cutting end of the instrument to follow a similar trajectory within the bone. The three-dimensional shape of the outer end 66, along with the size and configuration of the slot 64, therefore determines in three-dimensions the size and shape of the cut that will be made into the underlying bone surface.

FIGS. 6b and 7b show another arrangement, in which the slot 64′ takes the form of a cross. In the arrangement shown in 6c and 7c, the insert is provided with a combination of slots and holes 64″. In this particular case, the outer end 66″ is moulded or otherwise manufactured as a truncated cone shape, in order to constrain the cuts being made in the bone to that shape.

More generally, it will be understood that the insert may contain a variety of slots and holes, of any appropriate shape, according to the job in hand, and the outer end may also be of any desired camming profile. Conveniently, a variety of inserts having different slot configurations and camming profiles are provided for each possible tube size and shape, allowing the surgeon to select not only an appropriate tube but also an appropriate insert depending upon the procedure to be undertaken.

Because of the physical constraints provided by the slots and the camming effect of the outer end surface, the surgeon is prevented from inadvertently cutting away bone outside the area permitted by the chosen slot configuration. The surgeon is also prevented from cutting too deeply.

As is shown in FIG. 2, the longitudinal positioning of the insert within the tube is defined by means of an annular stop 22 formed around the inner tube surface adjacent the end that, in use, is secured to the bone. To provide further flexibility in longitudinal positioning, an annular spacer 24 may be provided which sits between the stop 22 and the end of the insert. A variety of spacers, of differing thicknesses, may be provided allowing the surgeon flexibility in selecting the longitudinal position of the insert within the tube.

In use, the surgeon selects an appropriate tube, insert and stop, secures the end of the tube to the bone surface to be prepared, by hammering or otherwise, and then inserts the cutting instrument through the slot, using the slot edges to cut against and the camming action of the outer end to define the cutting depth. Preferably, for additional security, the bone and the tube are both clamped separately using a standard surgical clamping arrangement (not shown).

Where additional clamps are provided, the system may also be used for surgical operations on body parts other than bone, for example on ligaments. The instruments being guided by the slot 64 will normally be cutting instruments, but could, alternatively, be any other type of surgical or investigative instrument requiring accurate positioning in three-dimensions.

The tubes may include fiducial points (not shown) allowing them to be tracked and located in three-dimensions by any standard surgical navigation system. The tubes may be used with radiolucent centring guides to allow the tubes to be accurately positioned under fluoroscopic guidance. External attachment means (not shown) may also be provided allowing the tubes to be easily and precisely clamped to the table, and/or to the patient, as the procedure demands.

Turning now to FIGS. 8 and 9, it will be described bow the preferred embodiment acts to protect the patient's tissue, and the operating theatre, from the debris created during the bone cutting procedure.

As is shown in FIGS. 8 and 9, the outer surface of the tube is formed with a series of outwardly-extending posts 80, spaced circumferentially around the tube, stretched over which there is an elastic strap or gaiter 82. Preferably, as is shown in FIG. 8, there are two sets of posts and two corresponding gaiters. Beneath each set of posts is a circumferential groove 84 in which the gaiter is arranged to be received when it is pushed off the adjacent posts.

Where the apparatus is to be used to prepare the surface of a joint for an implant, the surgeon first cuts back the joint capsule and tissues sufficiently to expose the underlying bone surface. As is shown in FIG. 10, clamps 100 are then attached to the joint capsule 102, and the joint capsule pulled up over the tube. This may normally be done by attaching tethers 104 to the clamps and then manually pulling on handles 106, attached to the tethers, in the direction of the arrows 108. The capsule is pulled up beneath the gaiter (or beneath all of the gaiters if there are several of them). The gaiter is then pushed off its posts so that the capsule is trapped—in “purse string” fashion—between the gaiter and the groove in which it sits.

Once the joint capsule has been secured in that way, cutting can proceed without any danger of debris leaving the joint. Any debris created that cannot be removed by suction remains in the area of the joint, and is sealed back underneath the joint capsule when the capsule is sewn up. The joint itself, within the capsule, is quite capable of dealing with small amounts of debris without difficulty. The invention in its preferred embodiments thus protects from debris any surrounding exposed tissue. The surrounding tissue is easier to clean at the end of the procedure, and is less likely to become infected.

FIGS. 11a and 11b are, respectively, longitudinal and sectional views of an embodiment of the invention being used to prepare a knee joint for an implant. As may be seen, the knee surface 110 to be prepared is first exposed, and a tube 111 inserted through the capsule. The drawing shows the tube just before it is hammered into the bone surface. Once secured, the capsule is pulled underneath the gaiter 82, an appropriate insert (not shown) is located within the tube, and the bone may then be cut, as shown at 114, for receipt of the implant.

FIGS. 12a and 12b are, respectively, front and side views of the apparatus being used to prepare a femur for a hip replacement. Here, the tube 120 is sized so that the head 122 of the femur 124 fits within the end of the tube. The capsule 126 is pulled up around the tube, as before, and secured by the gaiter (not shown).

FIGS. 13a and 13b show how the apparatus may be used for the preparation of convex surfaces. FIG. 13a shows the configuration for machining the acetabalum. A tube 130 is seated on a marginal osteophytes, with the hip being dislocated anteriorly at a precise orientation. FIG. 13b shows a tube 134 seated on the osteophytes with the hip being dislocated posteriorly at a precise orientation. Here, the capsule is shown at 136.

Finally, FIG. 14 shows the procedure for knee and tibial preparation, particularly where the machining of the tibial plateau surface is required. In this case, the tube 140 is seated onto the tibia 142 at the precise angle θ required for the bone preparation. The capsule is shown at 144.

1. An apparatus for surgical instrument location comprising:
an open-ended tubes
and an insert for insertion into the tube,
the insert having a longitudinal aperture formed therein and the lateral edges of the tube defining an operational area for an instrument inserted through the aperture;
a clamping arrangement for clamping the tube; and,
a surgical navigation system for locating the position of the tube within a frame of reference.

2. An apparatus as claimed in claim 1 further including a stop on the tube for positive longitudinal location of the insert within the tube.

3. An apparatus as claimed in claim 2 further including a removable spacer between the stop and the insert.

4. An apparatus as claimed in claim 1 further including rotational location means for positive selected rotational location of the insert within the tube.

5. An apparatus as claimed in claim 4 in which the rotational location means comprises co-operating longitudinal grooves on the tube and longitudinally-extending lugs on the insert.

6. An apparatus as claimed in claim 4 in which the rotational location means comprises co-operating longitudinally-extending lugs on the tube and longitudinal grooves on the insert.

7. An apparatus as claimed in claim 4 in which the rotational location means comprises co-operating longitudinal grooves on the tube and longitudinally-extending members on a head of the insert and on an outwardly-extending rim of the tube.

8. An apparatus as claimed in claim 1 in which the tube has securing means at a patient end thereof, for securing the tube to a patient.

9. An apparatus as claimed in claim 8 in which the securing means comprise teeth for securing the tube to the bone of a patient.

10. An apparatus as claimed in claim 9 in which the teeth are sprung.

11. An apparatus as claimed in claim 8 in which the securing means are formed by a sharpened edge of a tube wall, at a patient end thereof.

12. An apparatus as claimed in claim 1 further including an elastic gaiter, surrounding the tube, for holding a joint capsule of a patient.

13. An apparatus as claimed in claim 12 further including holding means for holding the gaiter away from an outer wall of the tube.

14. An apparatus as claimed in claim 13 in which the holding means comprise posts, outwardly extending from the outer wall.

15. An apparatus as claimed in claim 12 further including an annular groove, around the tube, a joint capsule being held in use between the gaiter and the groove.

16. An apparatus as claimed in claim 1 in which the insert has a shaped outer end which, in use, acts as a camming surface for a cam follower on an instrument positioned within the aperture, thereby varying the longitudinal position of the instrument within the tube as the instrument is moved laterally across the operational area.

17. An apparatus as claimed in claim 1 in which the clamping arrangement clamps the tube to a patient.

18. An apparatus as claimed in claim 1 in which the clamping arrangement clamps the tube to a patient table.

19. An apparatus as claimed in claim 1 in which the tube includes fiducial points.

20. A surgical instrument location system comprising:
(a) a plurality of open-ended tubes of differing sizes or cross sections;
(b) a corresponding plurality of inserts for insertion into the tubes, each insert having a longitudinal aperture formed therein, and the lateral edges of the tube defining an operational area for an instrument inserted through the aperture.

21. A surgical instrument location system as claimed in claim 20 further including a plurality of different selectable inserts for each tube.

22. A surgical instrument location system as claimed in claim 20 further including stops on the tubes for positive longitudinal location of the inserts within the tubes, the system further including a plurality of selectable spacers, of various sizes, for positioning between a stop on a selected tube and a selected insert.

23. A surgical instrument location system as claimed in claim 20 in which the tubes include fiducial points allowing tracking of tube position by a surgical navigation system.

24. A method of locating a surgical instrument comprising the steps of:
a) using a surgical navigation system to locate a tube within a frame of reference, the tube being elongate and open-ended and having within it an insert with a longitudinal aperture having lateral edges;
b) clamping the tube and locating its position using a surgical navigation system; and,
c) inserting a surgical instrument through the aperture, the lateral edges of the aperture defining the instrument location.

25. A method as claimed in claim 24 further including the step of clamping the tube to a patient.

26. A method as claimed in claim 24 further including the step of clamping the tube to a patient table.

27. A method of surgery as claimed in claim 26 further including the step of carrying out surgery with the instrument inserted through the aperture.
